(19) United States
(12) Patent Application Publication
(10) Pub. No.: US 2025/0017733 A1
(43) Pub. Date: Jan. 16, 2025

(54) INTER-LEAFLET PATCH

(71) Applicant: EDWARDS LIFESCIENCES CORPORATION, Irvine, CA (US)

(72) Inventors: Tal Sheps, Givat Shmuel (IL); Yaron Herman, Givat Ada (IL); Aviv Galon, Tel Aviv (IL); Meir Abucasis, Caesarea (IL); Noam Nir, Pardes-Hanna (IL)

(21) Appl. No.: 18/901,376

(22) Filed: Sep. 30, 2024

Related U.S. Application Data
(63) Continuation of application No. PCT/US2023/018493, filed on Apr. 13, 2023.
(60) Provisional application No. 63/373,159, filed on Aug. 22, 2022.
(60) Provisional application No. 63/331,771, filed on Apr. 15, 2022.

(51) Int. Cl. A61F 2/24

(52) U.S. Cl. CPC A61F 2/246; A61F 2/2466; A61F 2220/0008; A61F 2230/005; A61F 2230/0052

(57) ABSTRACT

For repairing a valve situated between an atrium and a ventricle of a heart of a subject, and having a first leaflet and a second leaflet, apparatus is provided that includes an implant and a delivery tool. The implant includes a flexible patch defining slots along an edge of the patch; a first anchor; and a second anchor. The delivery tool is adapted to transluminally deliver the implant to the heart; and implant the implant in the ventricle by securing the patch against a ventricular surface of the first leaflet by anchoring the first anchor to the first leaflet, and against a ventricular surface of the second leaflet by anchoring the second anchor to the second leaflet, such that (i) chordae of the valve extend through the slots, and (ii) the patch bridges a gap between the first leaflet and the second leaflet. Other embodiments are also disclosed.

### CROSS-REFERENCE TO RELATED APPLICATIONS

This present application is a continuation of International Patent Application PCT/US2023/018493, filed Apr. 13, 2023, which claims the benefit of U.S. Patent Application No. 63/331,771, filed Apr. 15, 2022, and of U.S. Patent Application No. 63/373,159, filed Aug. 22, 2022, the entire disclosures all of which are incorporated by reference for all purposes.

Each of the above applications is incorporated herein by reference.

### TECHNICAL FIELD

Some applications of the present invention relate in general to bridging a gap between leaflets of a tricuspid valve. More specifically, some applications of the present invention relate to transluminally implanting such a patch across the gap.

### BACKGROUND

The native heart valves (e.g., the aortic, pulmonary, tricuspid and mitral valves) serve critical functions in assuring the forward flow of an adequate supply of blood through the cardiovascular system. These heart valves can be rendered less effective by congenital malformations, inflammatory processes, infectious conditions or disease. Such damage to the valves can result in serious cardiovascular compromise or death. Treatment for such disorders can be done with the surgical repair or replacement of the valve during open heart surgery or with transcatheter transvascular techniques for introducing and implanting prosthetic devices in a manner that is much less invasive than open heart surgery.

Valve regurgitation occurs when the native valve fails to close properly and blood flows into an atrium from a ventricle during the systole phase of heart contraction. Valve regurgitation is the most common form of valvular heart disease.

### SUMMARY OF THE INVENTION

Dilation of the annulus around the tricuspid valve of a subject may lead to regurgitation due to a lack of coaptation between leaflets of the tricuspid valve during ventricular systole. Some applications of the present application relate to, inter alia, to bridging a gap between two leaflets of the tricuspid valve. For some such applications, this may effectively bicuspidize the tricuspid valve.

A patch may be transluminally attached to two leaflets, such that the patch spans the gap between the leaflets.

For some applications, a patch of an implant is secured to a first leaflet of the tricuspid valve using a first anchor of the implant, and the patch is also secured to a second leaflet of the tricuspid valve using a second anchor of the implant, such that the patch spans a gap between the first and second leaflets. For some applications, a delivery tool is configured to transluminally deliver and implant the implant within the heart. For example, the delivery tool may be adapted to position the patch over the gap between the leaflets, and then transluminally implant the anchors through the patch and into tissue of the leaflets, such that the patch becomes implanted at the valve.

For some applications, the patch is secured to an atrial surface of the tricuspid valve.

For some applications, the patch is secured to a ventricular surface of the leaflets, such that during ventricular systole, the patch becomes pressed against the ventricular surface of the leaflets. For some such applications, the patch may define multiple slots along the edge of the patch, in order to allow chordae of the heart to be received within these slots—e.g. further securing the patch and/or avoiding interference with the chordae.

For some applications, a patch is transluminally attached to all three leaflets of a tricuspid valve. For some applications, such a patch closes or covers a gap between the three leaflets. For some applications, implanting the patch at the tricuspid valve may arrange the leaflets into more than one orifice via which, during ventricular diastole, blood can flow into the ventricle. Each of these orifices may, therefore, serve as a pseudo-valve. For example, three discrete orifices may be created by implantation of the patch.

For some applications, at least one anchor is driven through each leaflet, in order to secure the patch to the valve.

There is therefore provided, in accordance with some applications of the present invention, a method of repairing a heart of a subject.

For some applications, the method includes transluminally delivering an implant to the heart, the implant including a patch, a first anchor, and a second anchor.

For some applications, the method includes:
using the first anchor, securing the patch against a surface of a first leaflet of a tricuspid valve of the heart; and/or
using the second anchor, securing the patch against a surface of a second leaflet of the tricuspid valve such that the patch bridges a gap between the first leaflet and the second leaflet.

For some applications, securing the patch against the surface of the second leaflet includes:
driving the second anchor through the second leaflet, such that a cord extends, from the second anchor, through the second leaflet, and towards the patch, and subsequently, advancing the patch over and along the cord toward the second anchor.

For some applications:
the implant further includes a coaptation body attached to the patch, and
securing the patch against the surface of the second leaflet includes securing the patch such that the coaptation body extends into the gap.

For some applications:
the patch defines a patch plane, and
securing the patch such that the coaptation body extends into the gap includes securing the patch such that the coaptation body extends orthogonally away from the patch plane and into the gap.

For some applications:
transluminally delivering the implant to the heart includes transluminally delivering the implant to the heart while the coaptation body is in a compressed state, and
the method further includes expanding the coaptation body within the heart.

For some applications, expanding the coaptation body within the heart includes inflating the coaptation body within the heart.

For some applications:
transluminally delivering the implant to the heart includes transluminally delivering the implant to the heart while the coaptation body is constrained in the compressed state, and
expanding the coaptation body within the heart includes releasing the coaptation body to automatically expand within the heart.

For some applications:
the coaptation body is a panel, and
securing the patch such that the coaptation body extends into the gap includes securing the patch such that the panel extends into the gap.

For some applications:
the coaptation body is a spacer, and
securing the patch such that the coaptation body extends into the gap includes securing the patch such that the spacer extends into the gap.

For some applications:
the coaptation body is a block, and
securing the patch such that the coaptation body extends into the gap includes securing the patch such that the block extends into the gap.

For some applications:
securing the patch against the surface of the first leaflet includes driving the first anchor through the first leaflet, and
securing the patch against the surface of the second leaflet includes driving the second anchor through the second leaflet and into tissue of the second leaflet.

For some applications, the method includes covering the gap with the patch without eliminating the gap.

For some applications, the method includes covering the gap with the patch such that the tricuspid valve is bicuspidized.

For some applications:
the surface of the first leaflet is an atrial surface of the first leaflet, the surface of the second leaflet is an atrial surface of the second leaflet, and
securing the patch against the surface of the second leaflet includes securing the patch against the atrial surface of the second leaflet such that, within a right atrium of the heart, the patch bridges the gap between the first leaflet and the second leaflet.

For some applications:
the surface of the first leaflet is a ventricular surface of the first leaflet, the surface of the second leaflet is a ventricular surface of the second leaflet, and
securing the patch against the surface of the second leaflet includes securing the patch against the ventricular surface of the second leaflet.

For some applications:
the patch defines a plurality of slots along an edge of the patch, and
securing the patch against the ventricular surface of the second leaflet includes securing the patch against the ventricular surface of the second leaflet such that a chorda of the valve is disposed within a slot of the plurality of slots.

For some applications:
the first anchor is a first toggle anchor,
the second anchor is a second toggle anchor, and
securing the patch against the surface of the first leaflet includes driving the first toggle anchor through the first leaflet such that the first toggle anchor becomes disposed against an opposite surface of the first leaflet, and
securing the patch against the surface of the second leaflet includes driving the second toggle anchor through the second leaflet such that the second toggle anchor becomes disposed against an opposite surface of the second leaflet.

For some applications:
the first toggle anchor is attached to the patch via a first cord,
the second toggle anchor is attached to the patch via a second cord,
driving the first toggle anchor through the first leaflet includes driving the first toggle anchor through the first leaflet such that the first cord extends, from the first anchor, through the first leaflet, to the patch, and
driving the second toggle anchor through the second leaflet includes driving the second toggle anchor through the second leaflet such that the second cord extends, from the second anchor, through the second leaflet, to the patch.

For some applications, securing the patch against the surface of the second leaflet includes securing the patch against the surface of the second leaflet such that the patch covers a commissure that is common to the first leaflet and the second leaflet.

For some applications, securing the patch against the surface of the second leaflet includes securing the patch against the surface of the second leaflet such that the patch does not contact a third leaflet of the tricuspid valve.

For some applications, securing the patch against the surface of the second leaflet includes securing the patch against the surface of the second leaflet such that, during ventricular systole, a lip of the patch contacts a third leaflet of the tricuspid valve.

For some applications, securing the patch against the surface of the second leaflet includes securing the patch against the surface of the second leaflet such that the lip of the patch extends into a right ventricle of the heart.

For some applications, the implant further includes a third anchor, and the method further includes:
using the third anchor, securing the patch against a surface of a third leaflet of the tricuspid valve such that the patch bridges a gap between the first leaflet, the second leaflet, and the third leaflet.

For some applications, securing the patch against the surface of the third leaflet includes securing the patch against the surface of the third leaflet such that the first leaflet, the second leaflet, and the third leaflet form more than one discrete orifice via which, during ventricular diastole, blood can flow into the ventricle.

For some applications, securing the patch against the surface of the third leaflet includes securing the patch against the surface of the third leaflet such that the leaflets form three discrete orifices at the valve.

There is further provided, in accordance with some applications of the present invention, an apparatus for repairing a valve of a heart, the heart having an atrium, a ventricle, the valve disposed therebetween, the apparatus including an implant. The implant may include a flexible patch, a first anchor, and a second anchor.

For some applications, the apparatus further comprises a delivery tool adapted to:
position the patch in the heart, and/or
secure the patch against a surface of a first leaflet of the valve using the first anchor, and against a surface of a second leaflet of the valve using the second anchor, such that the patch bridges a gap between the first leaflet and the second leaflet.

For some applications:
the delivery tool further includes a reference support adapted to apply a reference force against an opposing surface of the first leaflet, and
the delivery tool is adapted to secure the patch against the surface of the first leaflet by driving the first anchor through the first leaflet while the reference support is pressed against the opposing surface of the first leaflet.

For some applications, the patch is self-expandable, such that the patch is adapted to be transluminally delivered, via the delivery tool, in a constrained state, and is biased to transition, from the constricted state, to a deployed state in which it is dimensioned to bridge the gap.

For some applications:
the surface of the first leaflet is an atrial surface of the first leaflet,
the surface of the second leaflet is an atrial surface of the second leaflet, and
the delivery tool is adapted to secure the patch against the atrial surface of the first leaflet and the atrial surface of the second leaflet such that the patch bridges the gap.

For some applications:
the surface of the first leaflet is a ventricular surface of the first leaflet,
the surface of the second leaflet is a ventricular surface of the second leaflet, and
the delivery tool is adapted to secure the patch against the ventricular surface of the first leaflet and the ventricular surface of the second leaflet such that the patch bridges the gap.

For some applications:
the patch defines a plurality of slots along an edge of the patch, and
the delivery tool is adapted to secure the patch against the ventricular surface of the second leaflet such that a chorda of the valve is disposed within a slot of the plurality of slots.

For some applications:
the first anchor is a first toggle anchor,
the second anchor is a second toggle anchor, and
the delivery tool is adapted to secure the patch against the surface of the first leaflet by driving the first toggle anchor through the first leaflet such that the first toggle anchor becomes disposed against an opposite surface of the first leaflet, and
the delivery tool is adapted to secure the patch against the surface of the second leaflet by driving the second toggle anchor through the second leaflet such that the second toggle anchor becomes disposed against an opposite surface of the second leaflet.

For some applications:
the first toggle anchor is attached to the patch via a first cord,
the second toggle anchor is attached to the patch via a second cord.

For some applications:
the gap is a gap between the first leaflet, the second leaflet, and the third leaflet,
the implant further includes a third anchor,
the delivery tool is further adapted to secure the patch against a surface of a third leaflet of the valve using the third anchor, such that the patch bridges the gap between the first leaflet, the second leaflet, and the third leaflet.

For some applications, the delivery tool is adapted to secure the patch against the surface of the third leaflet such that the first leaflet, the second leaflet, and the third leaflet form more than one discrete orifice via which, during ventricular diastole, blood can flow into the ventricle.

For some applications, the delivery tool is adapted to secure the patch against the surface of the third leaflet such that the leaflets form three discrete orifices at the valve.

For some applications, the patch includes a flexible sheet.

For some applications, the flexible sheet is impermeable.

For some applications, the flexible sheet is semi-impermeable.

For some applications, the flexible sheet includes a fabric.

For some applications, the flexible sheet includes a film.

For some applications, the patch includes a frame, adapted to support the flexible sheet.

For some applications, the frame extends along a perimeter of the flexible sheet.

For some applications, the delivery tool is adapted to deliver the implant to the heart, and includes:
a shaft, and
a first channel and a second channel, coupled to the shaft, each of the first and second channels adapted to house a respective one of the first and second anchors during delivery of the implant to the heart.

For some applications, the delivery tool has a delivery state in which the delivery tool is adapted to deliver the implant to the heart, and in which:
the channels are positioned medially, and
the patch is wrapped around a distal end of the central shaft, such that the patch forms a cup-like shape around the channels and the shaft.

For some applications:
the delivery tool has a deployment state in which the delivery tool is configured to anchor the first and second anchors to the first and second leaflets, and
the delivery tool is transitionable from the delivery state toward the deployment state by deflecting the first and second channels laterally with respect to the shaft.

For some applications, for each of the first anchor and the second anchor, the respective channel has an axial slit that accommodates a cord extending from the anchor to the patch.

For some applications:
the delivery tool is adapted to secure the patch against the surface of the first leaflet and the surface of the second leaflet by:
positioning the first channel against the first leaflet and second channel against the second leaflet, and
advancing each of the first and second anchors out of the respective channel and through the respective leaflet.

For some applications, the apparatus is configured such that, for each of the first and second anchors, advancing of the anchor out of the respective channel is facilitated by the respective cord sliding along the respective axial slit.

There is further provided, in accordance with some applications of the present invention, a method for repairing a heart of a subject, the heart having an atrium, a ventricle, and a valve therebetween.

The method may include transluminally attaching a first anchor to a first leaflet of the valve while the first anchor is attached to a first cord, and/or transluminally attaching a second anchor to a second leaflet of the valve while the second anchor is attached to a second cord.

The method may further include, while a patch is threaded onto the first cord and the second cord, transluminally advancing the patch over and along the first and second cords towards the valve.

The method may further include subsequently securing the patch to a surface of the first leaflet and a surface of the second leaflet by fastening the patch to the first and second cords.

For some applications, the first and second anchors are attached to the first and second leaflets from the ventricle (e.g. are advanced through a ventricular surface of the leaflets).

For some applications, the first and second anchors are attached to the first and second leaflets from the atrium (e.g. are advanced through an atrial surface of the leaflets).

For some applications, the valve is a bicuspid valve, and securing the patch to the surface of the first leaflet and the surface of the second leaflet includes securing the patch to the surface of the first leaflet and the surface of the second leaflet of the bicuspid valve.

For some applications, the valve is a tricuspid valve, and securing the patch to the surface of the first leaflet and the surface of the second leaflet includes securing the patch to the surface of the first leaflet and the surface of the second leaflet of the tricuspid valve.

For some applications, the method further includes:
transluminally attaching a third anchor to a third leaflet of the valve while the third anchor is attached to a third cord,
while the patch is threaded onto the third cord, transluminally advancing the patch over and along the third cord towards the valve, and
securing the patch to the third leaflet by fastening the patch to the third cords.

For some applications:
the ventricle is a right ventricle,
the surface of the first leaflet is a ventricular surface of the first leaflet,
the surface of the second leaflet is a ventricular surface of the second leaflet, and
securing the patch to the surface of the second leaflet includes securing the patch to the surface of the second leaflet such that, within the right ventricle, the patch bridges the gap between the first leaflet and the second leaflet.

For some applications:
the patch defines a plurality of slots along an edge of the patch, and
securing the patch to the surface of the second leaflet includes securing the patch to the surface of the second leaflet such that, within the right ventricle, a chorda of the valve is disposed within a slot of the plurality of slots.
There is further provided, in accordance with some applications, a method of repairing a valve of a heart of a subject, the method including:
transluminally delivering an implant to the heart, the implant including a patch, a coaptation body attached to the patch, and an anchor;
securing the patch against a surface of a first leaflet of the valve and against a surface of a second leaflet of the valve using at least the anchor, such that the coaptation body extends between the first leaflet and the second leaflet.

For some applications:
the valve is disposed between an atrium and a ventricle of the heart,
securing the patch against the surface of the first leaflet and against the surface of the second leaflet such that the coaptation body extends between the first leaflet and the second leaflet, includes securing the patch against an atrial surface of the first leaflet and against an atrial surface of the second leaflet such that the coaptation body extends between the first leaflet and the second leaflet toward the ventricle.

For some applications, the valve is a tricuspid valve, and securing the patch to the surface of the first leaflet of the valve includes securing the patch to a surface of a first leaflet of the tricuspid valve.

For some applications:
the patch defines a patch plane, and
securing the patch to the surface of the first leaflet and the surface of the second leaflet includes securing the patch such that the coaptation body extends orthogonally away from the patch plane and towards a ventricle downstream of the valve.

For some applications:
the coaptation body is a panel, and
securing the patch to the surface of the first leaflet and the surface of the second leaflet includes securing the patch such that the panel extends into a gap between the first leaflet and the second leaflet.

For some applications:
the coaptation body is a spacer, and
securing the patch to the surface of the first leaflet and the surface of the second leaflet includes securing the patch such that the spacer extends into a gap between the first leaflet and the second leaflet.

For some applications:
the coaptation body is a block, and
securing the patch such to the surface of the first leaflet and the surface of the second leaflet includes securing the patch such that the block extends into a gap between the first leaflet and the second leaflet.

For some applications:
transluminally delivering the implant to the heart includes transluminally delivering the implant to the heart while the coaptation body is in a compressed state, and
the method further includes expanding the coaptation body within the heart.

For some applications, expanding the coaptation body within the heart includes inflating the coaptation body within the heart.

For some applications:
transluminally delivering the implant to the heart includes transluminally delivering the implant to the heart while the coaptation body is constrained in the compressed state, and
expanding the coaptation body within the heart includes releasing the coaptation body to automatically expand within the heart.

There is further provided, in accordance with some applications, an apparatus for repairing a valve of a heart, the heart having an atrium, a ventricle, the valve disposed therebetween, the apparatus including:
an implant, including:
a flexible patch;
a coaptation body, attached to the patch;
a first anchor; and
a second anchor; and
a delivery tool adapted to:
position the patch in the heart, and
secure the patch against a surface of a first leaflet of the valve using the first anchor, and against a surface of a second leaflet of the valve using the second anchor, such that the coaptation body extends between the first leaflet and the second leaflet.

For some applications, the coaptation body has a surface configured to encourage adhesion of the first leaflet and the second leaflet thereto.

For some applications, the implant includes a resilient frame that defines at least part of the patch and at least part of the coaptation body.

For some applications, the coaptation body is a block.

For some applications, the implant includes a sheet that defines a surface of the patch and a surface of the coaptation body.

For some applications, the coaptation body includes a resilient frame.

For some applications, the patch includes a sheet that covers frame.

For some applications, the coaptation body defines a hollow therein.

For some applications, the delivery tool is configured to inflate the coaptation body with a liquid.

For some applications, the coaptation body is configured to encourage fibrosis inside the hollow.

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

### BRIEF DESCRIPTION OF THE DRAWINGS

FIGS. 1-4 show schematic illustrations of various techniques for bridging a gap between leaflets of a tricuspid valve;

FIGS. 5A-B and 6A-C are schematic illustrations of a system comprising an implant for bridging a gap between leaflets of a tricuspid valve, and a delivery tool for delivering and implanting the implant;

FIG. 7 is a schematic illustration of a technique for implanting a patch at a tricuspid valve; and

FIG. 8 is a schematic illustration of a technique for implanting a patch having a coaptation body attached thereto to a tricuspid valve, such that the coaptation body extends into a gap between the leaflets.

### DETAILED DESCRIPTION OF EMBODIMENTS

Dilation of the annulus around the tricuspid valve of a subject may lead to regurgitation due to a lack of coaptation between leaflets of the tricuspid valve during ventricular systole. Some applications of the present application relate to, inter alia, bridging a gap between two leaflets of the tricuspid valve. For some such applications, this may effectively bicuspidize the tricuspid valve—e.g. by forming a unified leaflet that includes the two leaflets.

For some applications, in order to bridge the gap, a patch of an implant is secured across the gap. For some such applications, the patch is configured to encourage adhesion of the leaflets thereto and/or to encourage tissue growth between the leaflets and the implant. This may, in effect, advantageously fuse the leaflets together over time, e.g. effectively bicuspidizing, or partly bicuspidizing, the valve.

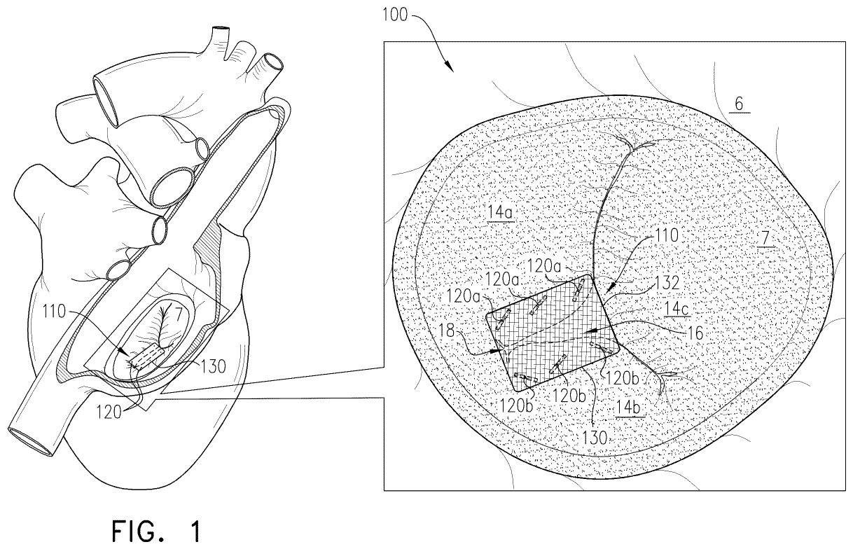

Reference is now made to FIG. 1, which is a schematic illustration of a system 100 comprising an implant 110 for bridging a gap 16 between leaflets of a tricuspid valve 7. Implant 110 comprises a patch 130, and tissue anchors 120 via which the patch is securable to leaflets 14a and 14b of valve 7.

Patch 130 may be transluminally attached to two leaflets 14a and 14b of the valve, such that the patch spans gap 16 between the leaflets.

For some applications, patch 130 is secured to first leaflet 14a of the tricuspid valve using at least one first anchor 120a of the implant, and the patch is also secured to second leaflet 14b of the tricuspid valve using at least one second anchor 120b of the implant, such that the patch spans gap 16 between the first and second leaflets. In the example shown, implant 110 comprises three first anchors 120a and three second anchors 120b.

For some applications, and as shown, anchors 120 are toggle anchors, and are anchored by driving the anchors through the leaflet such that the toggle anchor becomes disposed against the opposite surface of that leaflet—e.g. the surface opposite that against which patch 130 is disposed.

For some applications, such toggle anchors are attached to patch 130 via a cord (not visible), such that after anchoring, the cord extends, from the anchor, through the leaflet, to the patch.

For some applications, a delivery tool (not shown) is configured to transluminally deliver and implant implant 110 within the heart. For example, the delivery tool may be adapted to position the patch over the gap between the leaflets, and then subsequently transluminally implant the anchors through the patch and into tissue of the leaflets, such that the patch becomes implanted at the valve. For some applications, the delivery tool may be adapted to anchor one or more of first anchors 120a (e.g. while the patch remains in a delivery configuration), to subsequently deploy and/or position the patch over the gap 16, and to subsequently anchor one or more of second anchors 120b. For some applications, the delivery tool may be adapted to anchor all of the anchors of implant 110 prior to deploying/positioning patch 130.

For some applications, and as shown in FIG. 1, implant 110 is implanted such that patch 130 covers a commissure 18 that is common to leaflets 14a and 14b—e.g. obstructing blood from flowing through the commissure 18. For some such applications, and as shown in FIG. 1, implant 110 is implanted such that patch 130 extends sufficiently far along the respective lips of leaflets 14a and 14b such that, during ventricular systole, the patch 130 reaches the third leaflet 14c of the valve—e.g. such that a lip region 132 of the patch 130 coapts with a lip of leaflet 14c. Thus, for some applications, patch 130 effectively bicuspidizes the tricuspid valve—e.g. such that a unified leaflet is formed from leaflet 14a, patch 130, and leaflet 14b. As described hereinabove, patch 130 may be configured to encourage adhesion of leaflets 14a and 14b thereto and/or to encourage tissue growth between the leaflets and the implant. This may, in effect, advantageously fuse the leaflets together over time.

It is to be noted that, despite the implantation of implant 110, gap 16 may remain—e.g. as shown. That is, the effect of implanting patch 130 over gap 16 may reduce tricuspid regurgitation, without requiring leaflets 14a and 14b to be drawn together such that the gap is eliminated entirely. This may advantageously result in less tugging/tension on the leaflets compared with techniques in which the leaflets are drawn together. This may be particularly advantageous for valves having a large gap between the leaflets (e.g. as a result of a particularly dilated valve annulus).

Although not shown, for some applications another anchor (which may be similar to or different from anchors 120) may be used to anchor patch 130 in the vicinity of commissure 18—e.g. to the annulus behind the commissure.

For some applications, and as shown, patch 130 is secured to an upstream (e.g. atrial) surface of the leaflets of the tricuspid valve—e.g. with anchors 120 disposed against a downstream (e.g. ventricular) surface of the leaflets.

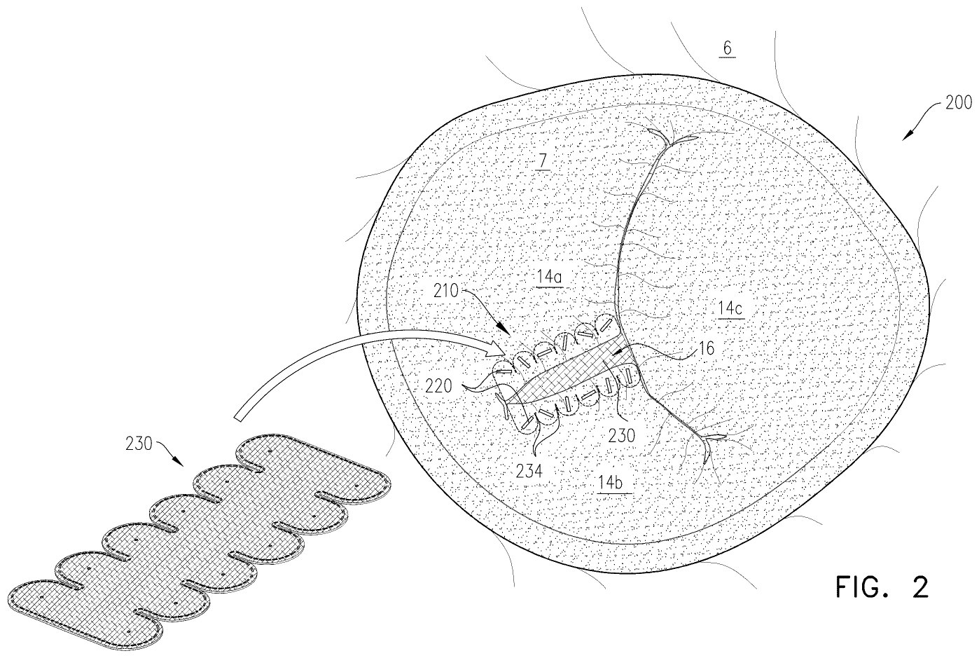

Reference is now made to FIG. 2, which is a schematic illustration of a system 200 that, similarly to FIG. 1, comprises an implant 210 for bridging a gap 16 between leaflets 14a and 14b of a tricuspid valve 7. Similarly to FIG. 1, implant 210 comprises a patch 230, and tissue anchors 220 via which the patch is securable to leaflets 14a and 14b of valve 7. For some applications, system 200 may be considered to be a variant of system 100, e.g. patch 230 may be considered to be a variant of patch 130 and/or anchors 220 may be considered to be variants, or substantially identical to, anchors 120.

As shown, patch 230 may be secured to a ventricular surface of the leaflets, such that during ventricular systole, the patch becomes pressed against the ventricular surface of the leaflets. For example, FIG. 2 shows an upstream perspective (e.g. a perspective from the right atrium 6 of the heart) of valve 7, with patch 230 disposed underneath the valve (e.g. within the ventricle). Anchors 220 are embodied as toggle anchors and are disposed against the atrial surface of leaflets 14a and 14b.

For some such applications, patch 230 defines slots 234 along one or more edges of the patch, in order to allow chordae of the heart to be received within these slots. This may facilitate placement of the patch against the surface of the leaflet (e.g. while avoiding interference with the chordae), and/or may further secure the implant in place.

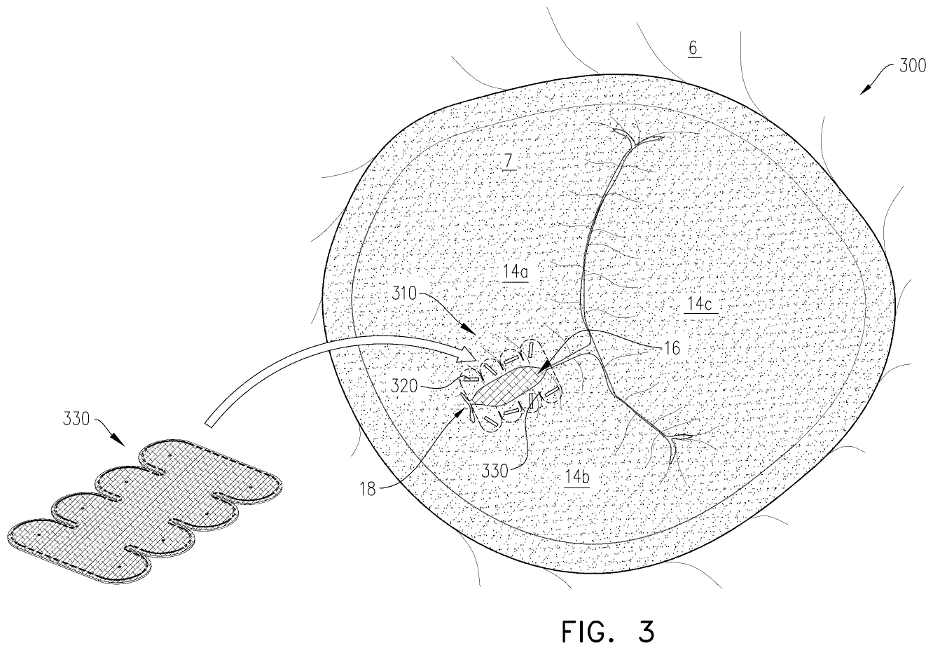

Reference is now made to FIG. 3, which is a schematic illustration a system 300 that, similarly to systems 100 and 200, comprises an implant 310 for bridging a gap 16 between leaflets 14a and 14b of a tricuspid valve 7. Similarly to implants 110 and 210, implant 310 comprises a patch 330, and tissue anchors 320 via which the patch is securable to leaflets 14a and 14b of valve 7. For some applications, system 300 may be considered to be a variant of system 200, e.g. patch 330 may be considered to be a variant of patch 230 (e.g. defining multiple slots and implantable at a ventricular surface of the leaflets) and/or anchors 320 may be considered to be variants, or substantially identical to, anchors 220.

As illustrated in FIG. 3, patch 330 may be dimensioned to extend only partway along the lips of leaflets 14a and 14b—e.g. without reaching leaflet 14c. For some applications, this may have a similar effect to patch 230 but may not lead to bicuspidization of the valve. For example, patch 330 may not extend along the entire coaptation line between leaflets 14a and 14b. This may be used for applications in which gap 16 does not extend along the entire coaptation line between the leaflets. For some applications, implant 310 may be implanted with patch 330 reaching (e.g. covering) commissure 18. For some applications, implant 310 may be implanted with patch 330 not reaching commissure 18.

For some applications, implant 310 (or a variant thereof) may be implanted with patch 330 against an atrial surface of the leaflets, rather than against a ventricular surface of the leaflets. For some applications, patch 330 (or a variant thereof) may not define slots. Similarly, patch 130 may be dimensioned to extend only partway along the lips of leaflets 14a and 14b.

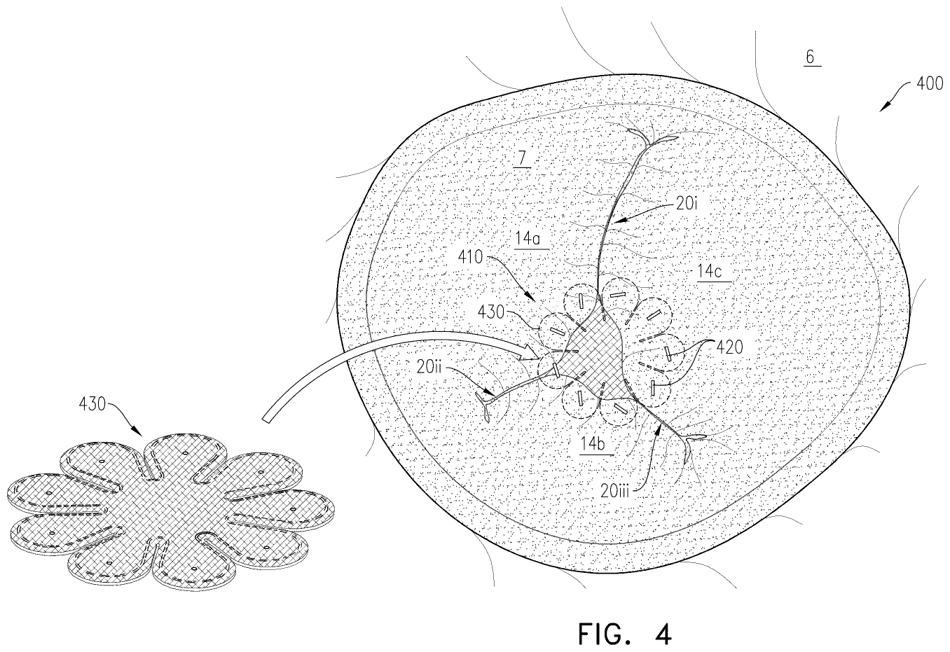

Reference is now made to FIG. 4, which is a schematic illustration of a system 400 comprising an implant 410 for reducing tricuspid regurgitation. Implant 410 comprises a patch 430, and tissue anchors 420 via which the patch 430 is securable to leaflets 14a, 14b, and 14c of valve 7.

As illustrated in FIG. 4, patch 430 may be transluminally attached to all three leaflets 14a, 14b, and 14c of the tricuspid valve 7, such that the patch 430 spans a gap between all three of the leaflets.

For some applications, patch 430 closes or covers the gap between the leaflets. For some applications, implanting patch 430 at tricuspid valve 7 may arrange the leaflets into more than one orifice via which, during ventricular diastole, blood can flow into the ventricle. Each of these orifices may, therefore, serve as a pseudo-valve. In the example shown, three discrete orifices 20i, 20ii, and 20iii have been created by implantation of patch 430.

For some applications, the patch 430 may be secured to the leaflets using anchors 420—e.g. as described with reference to anchors 120, 220, and 320 hereinabove (e.g. anchors 420 may be considered to be variants of these anchors, for example, the anchors may be toggle anchors). For some applications, patch 430 is secured to each of first leaflet 14a, second leaflet 14b, and third leaflet 14c using at least one anchor 420 of the implant for each leaflet, such that the patch spans gap 16 between the leaflets. In the example shown, multiple anchors are implanted at each leaflet.

As shown, patch 430 may be secured to a ventricular surface of the leaflets, such that during ventricular systole, the patch 430 becomes pressed against the ventricular surface of the leaflets. For example, and similarly to FIGS. 2 and 3, FIG. 4 shows an upstream perspective (e.g. a perspective from the right atrium 6 of the heart) of valve 7, with patch 430 disposed underneath the valve (e.g. within the ventricle). Anchors 420 are embodied as toggle anchors and are disposed against the atrial surface of leaflets 14a, 14b, and 14c.

For some such applications, and as described hereinabove with reference to FIG. 2, patch 430 defines slots along one or more edges of the patch 430, in order to allow chordae of the heart to be received within these slots. This may facilitate placement of the patch 430 against the surface of the leaflet (e.g. while avoiding interference with the chordae), and/or may further secure the implant in place. In the particular example shown, patch 430 is shaped generally as a circular flower, with the slots disposed between the petals of the flower. Further in the particular example shown, patch 430 has nine such petals and nine slots therebetween.

For some applications, implant 410 (or a variant thereof) may be implanted with patch 430 against an atrial surface of the leaflets, rather than against a ventricular surface of the leaflets. For some applications, patch 430 (or a variant thereof) may not define slots.

Similarly to as described hereinabove, patch 430 may be configured to encourage adhesion of leaflets 14a, 14b, and 14c thereto and/or to encourage tissue growth between the leaflets and the implant. This may, in effect, advantageously fuse a central part of the leaflets together over time.

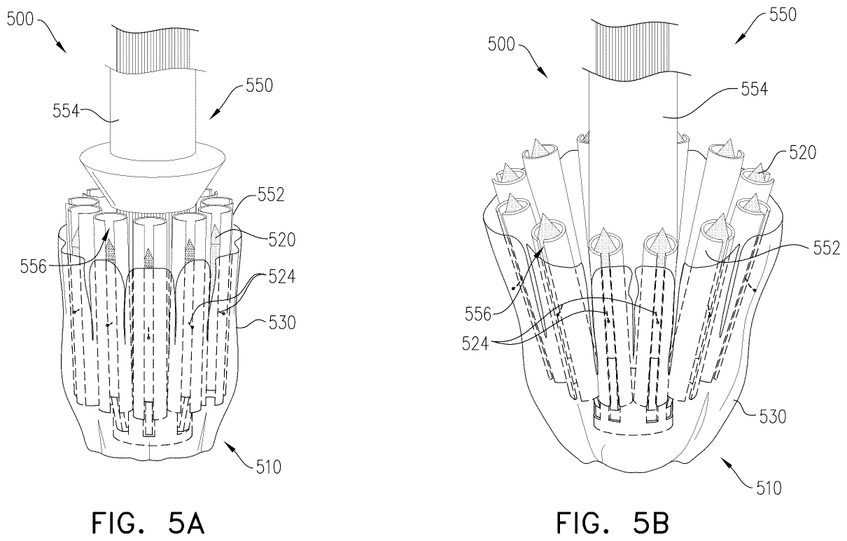

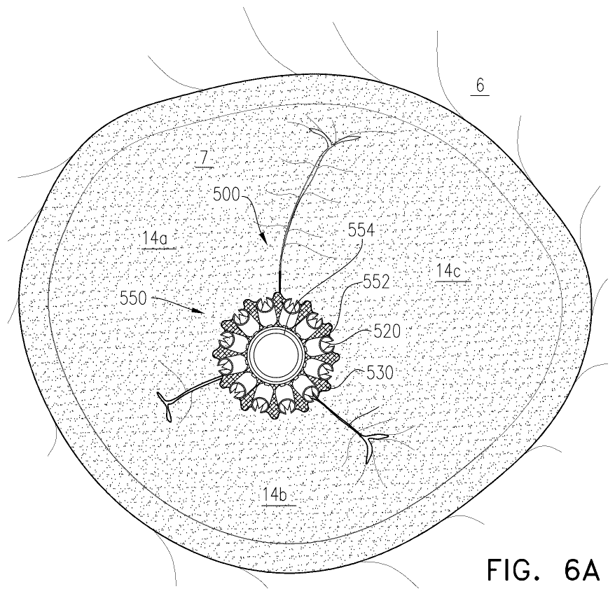

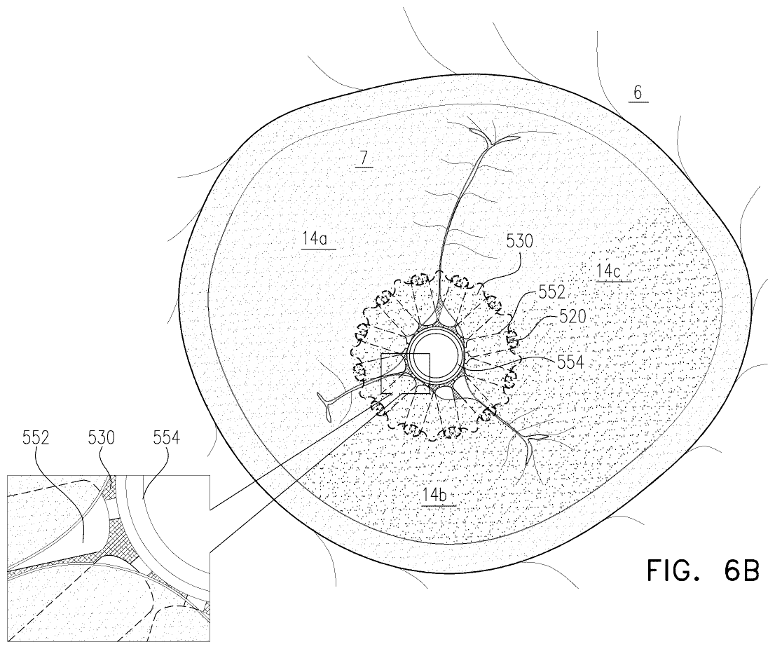

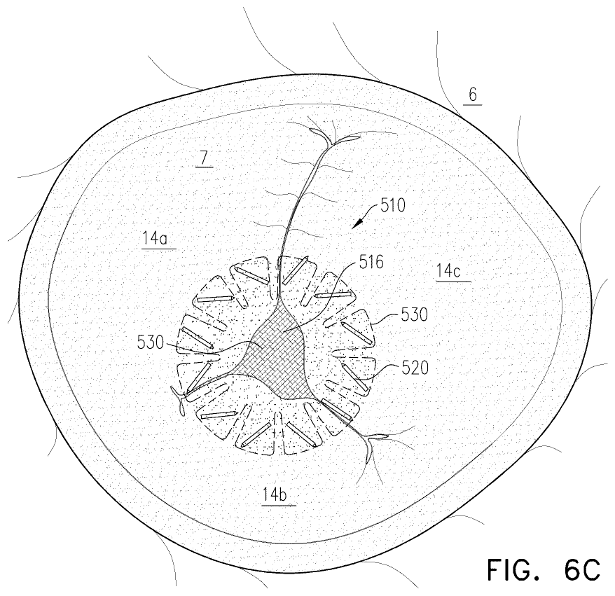

Reference is now made to FIGS. 5A-B, and 6A-C, which are schematic illustrations of a system 500 comprising an implant 510 for bridging a gap 516 between leaflets of a tricuspid valve 7, and a delivery tool 550 for delivering and implanting the implant. Similarly to FIGS. 1-4, implant 510 comprises a patch 530, and tissue anchors 520 via which the patch is securable to leaflets of the valve. As described hereinbelow, delivery tool 550 is typically adapted to implant patch 530 against a surface (e.g. a ventricular surface) of the leaflets. For some applications, and as described hereinabove, patch 530 may define a plurality of slots, for accommodation of chordae of the ventricle.

FIGS. 5A-6C show patch 530 somewhat resembling patch 430—e.g. a patch that is securable to all three leaflets of a tricuspid valve, and/or that resembles a circular flower. Patch 530 may be considered to be a variant of patch 430, but with twelve petals and slots rather than nine. However, it is to be understood that, for some applications, patch 530 may represent and/or may be considered to be a variant of any of patches 130, 230, 330, and/or 430. Similarly, tool 550, or a variant thereof, mutatis mutandis, may be used to implant any of patches 130, 230, 330, and/or 430.

For some applications, and as shown, anchors 520 are toggle anchors, and are anchored by driving the anchors through the leaflet such that the toggle anchor becomes disposed against the opposite surface of that leaflet—e.g. the surface of the leaflets opposite that against which patch 530 is disposed (e.g. the atrial surface).

For some applications, such toggle anchors are attached to patch 530 via a cord 524, such that after anchoring, the cord extends, from the anchor, through the leaflet, to the patch.

Delivery tool 550 comprises a central shaft 554, to which a plurality of channels 552 are attached. Each of channels 552 is typically adapted to house an anchor 520 during delivery of the implant to the heart. FIG. 5A shows delivery tool 550 in a delivery state in which channels 552 are positioned medially (e.g. relatively close to shaft 554), such that the distal region of the tool can be delivered to the heart, e.g. via an outer sheath. As shown, in the delivery state, patch 530 may be wrapped around a distal end of the delivery tool, such that the patch forms a cup-like shape around channels 552. Each channel 552 may have an axial slit 556 (e.g. facing laterally) that accommodates cord 524 extending from anchor 520 to connect the anchor to patch 530.

Delivery tool 550, with implant 510 therewith, may be advanced to the right atrium 6 (FIG. 6A). At this stage, delivery tool 550 may be transitioned into a partly open state—e.g. as shown. Alternatively, delivery tool 550 may remain in the delivery state at this stage.

Delivery tool 550 is then advanced into the right ventricle, such that the open end of each channel 552 faces upstream towards leaflets 14 (FIG. 6B). At this stage, and as shown by the transition between FIGS. 6A and 6B, delivery tool may be transitioned into a deployment state, in which the open ends of channels 552 are positioned more laterally (e.g. radially outward) from shaft 554. For example, in the deployment state of the delivery tool, the channels may extend obliquely to shaft 554, e.g. proximally and outwardly from a distal part of the shaft (FIG. 5B). For some applications, tool 550 may comprise a mechanical linkage that transitions the tool from the delivery state to the deployment state by deflecting the channels laterally away from shaft.

FIG. 5B also shows tool 550 in its deployment state. For some applications, and as shown in FIG. 5B, in this state the tips of anchors 520 may be exposed from channels 552, such that the tips of the anchors can protrude slightly into, and thereby grip, the leaflets. This may be useful to facilitate positioning and/or stabilization of the delivery tool with respect to the leaflets prior to and/or during deployment of the anchors. For some applications, the tips of anchors 520 may be exposed in this manner even in the delivery state. For some applications, the tips of anchors 520 are exposed only once the channels are positioned at the valve (e.g. with the open ends facing the leaflets)—e.g. as part of, or subsequently to, transitioning into the deployment state.

At this stage, patch 530 may remain wrapped around the channels, e.g. in the ventricle, downstream from the valve and the anchors.

For some applications, anchors 520 are then advanced out of their channels 552 and through the leaflets, such that at least one anchor is driven through each of leaflets 14a, 14b, and 14c. This may be facilitated by cords 524 sliding along axial slits 556 until reaching the open end of the channel. As described hereinabove, and as illustrated, anchors 520 may be toggle anchors, and can be advanced out of channels 552, through the leaflets, and into the right atrium, such that the cord extends through the leaflets, connecting the anchor to the patch.

For some applications, delivery tool 550 is then withdrawn through valve 7, through right atrium 6, and out of the heart—e.g. after being returned into its delivery state. For some applications, withdrawing the delivery tool allows a central part of the patch 530 to move toward planar alignment with the leaflets. As illustrated in FIG. 6C, the implanted implant 510 thus bridges gap 516 between leaflets 14a, 14b, and 14c.

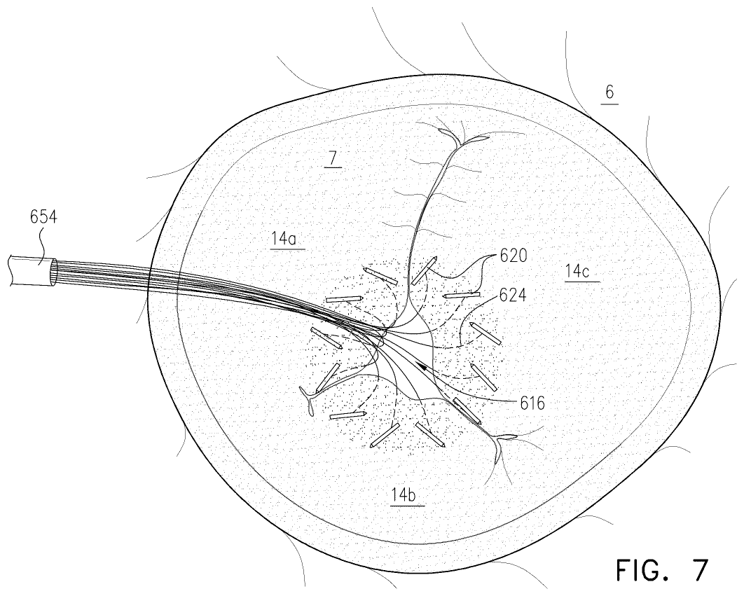

Reference is now made to FIG. 7, which is a schematic illustration of an alternative technique for implanting a patch across a valve 7.

For some applications, prior to implanting the patch, multiple anchors 620 are first anchored to (e.g. driven into) the leaflets of the valve. For some applications, this may be achieved using the apparatus and/or technique described hereinabove with reference to FIGS. 6A-C, mutatis mutandis. However, other apparatus and/or techniques may be alternatively or additionally used. Once anchors 620 have been anchored, cords 624, which are attached to the anchors, extend from the anchors, through right atrium 6, and out of the heart (e.g. out of the subject)—e.g. via a catheter 654. For some applications, and as shown, anchors 620 are disposed on an upstream (e.g. atrial) surface of leaflets 14, and cords 624 extend from the anchors, through the leaflets into the ventricle, turning upstream through a gap 616 between the leaflets, from where they extend through the atrium and out of the heart.

The patch (e.g. a patch similar to any of patches 130, 230, 330, 430 and/or 530), is then parachuted over cords 624, such that the patch slides over and along the cords, thereby following the path of the cords until it becomes disposed against a surface of the leaflets. In the example shown, the patch would be slid through gap 616, and pressed against the downstream (e.g. ventricular) surface of the leaflets. The patch may then be secured in place using stoppers (e.g. locks or crimps)—e.g. one per cord 624. Cords 624 may then be trimmed (e.g. just proximally from the stoppers).

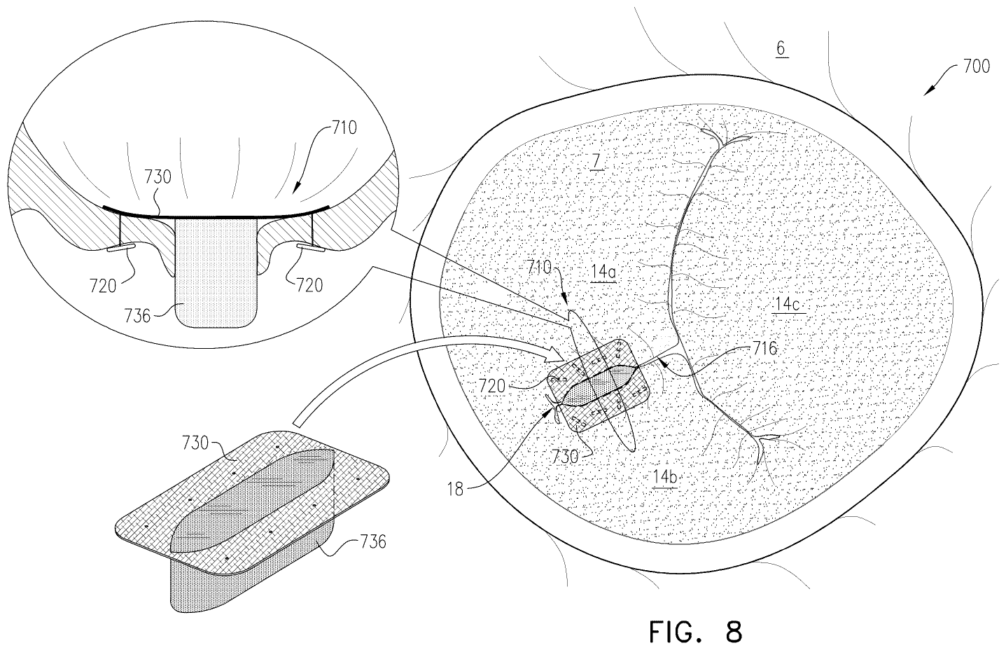

Reference is now made to FIG. 8, which is a schematic illustration of a system 700 comprising an implant 710 for reducing regurgitation between leaflets of a heart valve, such as a tricuspid valve 7. In addition to comprising a patch 730 (which may be considered to be a variant of any of the patches mentioned hereinabove), and tissue anchors 720 via which the patch is securable to leaflets 14a and 14b of the valve, implant 710 further comprises a coaptation body 736, attached to the patch, and adapted to extend from the patch between the leaflets (e.g. toward the ventricle), once the patch is secured to the leaflets. Positioning coaptation body 736 between the leaflets (e.g. in a gap 716 between the leaflets) may advantageously cause leaflets 14a and 14b to coapt around the coaptation body, thereby advantageously reducing the need to pull the leaflets towards each other. This may augment the advantageous effect, described hereinabove, of implanting a patch that bridges a gap between the leaflets.

It is to be noted that although patch 730 is shown as being a variant of patch 130 described hereinabove, it is to be understood that, for some applications, patch 730 may represent and/or may be considered to be a variant of any of patches 230, 330, 430 and/or 530, e.g. patch 730 may be define a plurality of slots, and/or may be anchored to three leaflets of a tricuspid valve of the heart, e.g. as described with reference to patch 430.

For some embodiments, coaptation body 736 may reduce atrially-directed forces experienced by leaflets 14a and 14b, and/or by patch 730 during ventricular systole of the heart. For example, at least some of the atrially-directed force experienced by the valve may be deflected laterally as the leaflets are urged into coaptation with coaptation body 736.

As shown, coaptation body 736 may extend orthogonally away from patch 730, such that when the patch is secured to leaflets 14a and 14b, the coaptation body extends, from the leaflets, and toward (e.g. into) the ventricle downstream of valve, such that the lips of the leaflets coapt (e.g. seal) around the coaptation body.

For some applications, implant 710 comprises a resilient frame that defines at least part of coaptation body 736. For some applications, the resilient frame defines at least part of patch 730 as well as at least part of the coaptation body. For some applications, the frame is covered by a sheet such as a fabric or a film. This sheet may belong to only the coaptation body, or may belong to both the coaptation body and the patch.

For some applications, coaptation body 736 is a panel that extends away from the patch (e.g. such that the panel and coaptation body collectively define a T-shape). The panel may be flat (e.g. rectangular, or another substantially two-dimensional shape) or may be curved. The panel may comprise a resilient frame (which may be a grid or trellis, or may merely extend around the perimeter of the panel). The frame may be covered on one or both sides by a sheet. For such applications, the contact (e.g. frictional contact) that leaflets 14a and 14b experience when coapting against the panel may be sufficient to reduce or eliminate regurgitation from the ventricle into atrium 6.

For some embodiments, coaptation body 736 is delivered to the heart in a compressed state (e.g. while constrained in the compressed state within a catheter of a delivery tool). For some applications, the coaptation body is biased to automatically expand once it is deployed out of the delivery tool, within the heart.

For some applications, coaptation body 736 is a block (e.g. a three-dimensional bulge), that may be hull-shaped and/or bulb shaped. Such a block may be advantageous for valves having a large gap between the leaflets (e.g. as a result of a particularly dilated valve annulus)—e.g. by filling in the gap, thereby allowing the leaflets to coapt around the coaptation body, rather than the leaflets having to extend to meet each other.

For some applications, the block includes at least part of a frame that automatically expands the block upon deployment within the heart. Alternatively, for some applications, the delivery tool is adapted to expand the coaptation body by inflating it within the heart, e.g. by filling a hollow defined by the coaptation body with a liquid.

For some embodiments, coaptation body is non-expandable.

For some applications, coaptation body 736 comprises a permeable sheet (e.g. a fabric) that surrounds a hollow (e.g. by being supported by a frame). For some such applications, the sheet allows blood to enter the hollow. For some such applications, the coaptation body (e.g. the sheet) is configured to encourage fibrosis inside the hollow.

For some applications, patch 730 and/or coaptation body 736 (e.g. a surface of the coaptation body against which leaflets 14a and 14b coapt) is configured to encourage adhesion of leaflets 14a and 14b thereto and/or to encourage tissue growth between the leaflets and the implant. This may, in effect, advantageously fuse the leaflets together over time, e.g. effectively bicuspidizing, or partly bicuspidizing, the valve.

For some applications, implant 730 comprises a sheet that defines both a surface of the patch and a surface of the coaptation body, e.g. the sheet providing a single surface that extends continuously over the patch and the coaptation body. For some applications, the entirety of implant 710 is coated with a continuous coat of a polymer. For some applications, patch 730 and coaptation body 736 are (e.g. the entirety of implant 710 is) molded as a single monolithic item.

For some applications, anchors 720 may be considered to be variants of any of the anchors described hereinabove, e.g. toggle anchors, pledget anchors, helical anchors or any other tissue anchors known in the art.

Reference is again made to FIGS. 1-8. Patches 130, 230, 330, 430, 530 and/or 730 may comprise a flexible sheet, such as a sheet of a fabric and/or a film. For some applications, the sheet may be impermeable, e.g. such that once the patch is implanted, the patch acts as a barrier at the gap. For some applications, the sheet may be semi-permeable—e.g. such that the patch only partially closes the gap. For some applications, the flexible sheet is supported by a frame. For some applications, such a frame may extend along a perimeter of the valve, e.g. as illustrated in FIGS. 2-4. However, it is to be understood that such a frame may be constructed to support the patch (e.g. a flexible sheet thereof) in any other configuration—e.g. including cross-struts and/or a lattice structure.

For some applications, the patch (e.g. the frame thereof) can be self-expanding, such that the patch can transition from a delivery state of the patch, in which the patch is constrained within a delivery tool (e.g. within a catheter), to a deployed state in which the patch assumes a more expanded configuration (e.g. by the frame pushing the sheet outwardly).

Reference is again made to FIGS. 1-8. For some applications, any of the anchors described herein (e.g. any of anchors 120, 220, 320, 420, 520, 620, or 720) may be pledget anchors, e.g. as described in US Patent Application Publication 2019/0254677 to Cahalane, which is incorporated by reference herein in its entirety. For some applications, any of the anchors described herein (e.g. any of anchors 120, 220, 320, 420, 520, 620, or 720) may be a variant of, or substantially identical to, any of the leaflet anchors described in PCT Publication WO 2022/101817, which is incorporated herein by reference, in its entirety.

Reference is again made to FIGS. 1-8. For some applications, during implantation of the implant at the valve (e.g. during driving of the anchors of the implant through the leaflets), a reference force is applied to the leaflets to facilitate the driving of the anchors through the leaflet—e.g. to prevent the leaflet from moving away from the anchors.

For some applications, such a reference force is achieved by positioning a reference support against an opposing surface of the leaflets. For example, in an application in which the anchors are driven into the leaflets from the atrium, during anchoring of the anchors into the leaflets, positioning the reference support against a ventricular surface of the leaflets may prevent the leaflets from moving away from the anchors and into the ventricle. For some applications, the reference support is expandable and/or deflectable e.g. is adapted to conform to the surface of the leaflets against which it is pressed, in order to provide sufficient force.

For some applications, reference support may be provided similarly to as described, mutatis mutandis, in International Patent Application PCT/IB2021/060436 to Tennenbaum et al., filed Nov. 11, 2021, which published as WO 2022/101817, which is incorporated herein by reference. For example, and as described in the aforementioned application, reference support may be embodied as a ledge-like element, similar to a “downstream support” described in Tennenbaum et al.

For applications in which the anchors are deployed through the leaflets from the ventricle (and into the atrium), a reference support may not be necessary, as the chordae tendineae may provide sufficient resistance, preventing the leaflets from moving atrially. However, a reference support may nonetheless be provided.

For some applications, the patch may be regularly or irregularly shaped in any manner, such as the circular flower-shaped patch 430 that may have multiple petals extending outward about a central circular region with a central origin, the rectangular or square-shaped patch 130 or the segmented or centipede shaped patch 230, to better facilitate coverage of the gap 16 and/or to facilitate anchoring, adhesion or connection with the leaflets.

Any of the systems, devices, apparatuses, etc. herein may be sterilized (e.g., with heat, radiation, ethylene oxide, hydrogen peroxide, etc.) to ensure they are safe for use with patients, and any of the methods herein can include sterilization of the associated system, device, apparatus, etc. (e.g., with heat, radiation, ethylene oxide, hydrogen peroxide, etc.) as one of the steps of the method.

### Example Applications (Some Non-Limiting Examples of the Concepts Herein are Recited Below)

Example 1. An apparatus for repairing a valve of a heart, the heart having an atrium, a ventricle, the valve disposed therebetween, the apparatus comprising:
an implant, comprising:
a flexible patch;
a first anchor; and
a second anchor; and
a delivery tool adapted to:
position the patch in the heart, and
secure the patch against a surface of a first leaflet of the valve using the first anchor, and against a surface of a second leaflet of the valve using the second anchor, such that the patch bridges a gap between the first leaflet and the second leaflet.

Example 2. The apparatus according to example 1, wherein the delivery tool is adapted to secure the patch against the surface of the first leaflet and the surface of the second leaflet such that the patch bridges the gap, without eliminating the gap.

Example 3. The apparatus according to any one of examples 1-2, wherein:
the valve is a tricuspid valve, and
the delivery tool is adapted to secure the patch against the surface of the first leaflet and the surface of the second leaflet such that the tricuspid valve is bicuspidized.

Example 4. The apparatus according to any one of examples 1-3, wherein the delivery tool is adapted to secure the patch against the surface of the first leaflet and the surface of the second leaflet such that the patch covers a commissure that is common to the first leaflet and the second leaflet.

Example 5. The apparatus according to any one of examples 1-4, wherein the apparatus is sterilized.

Example 6. The apparatus according to any one of examples 1-5, wherein the patch is configured to encourage adhesion of the first leaflet and the second leaflet thereto.

Example 7. The apparatus according to any one of examples 1-6, wherein:
the delivery tool further comprises a reference support adapted to apply a reference force against an opposing surface of the first leaflet, and
the delivery tool is adapted to secure the patch against the surface of the first leaflet by driving the first anchor through the first leaflet while the reference support is pressed against the opposing surface of the first leaflet.

Example 8. The apparatus according to any one of examples 1-7, wherein the implant further comprises a coaptation body, attached to the patch, and wherein the delivery tool is adapted to secure the patch such that the coaptation body extends into the gap.

Example 9. The apparatus according to example 8, wherein the coaptation body has a surface configured to encourage adhesion of the first leaflet and the second leaflet thereto.

Example 10. The apparatus according to example 8, wherein the implant comprises a resilient frame that defines at least part of the patch and at least part of the coaptation body.

Example 11. The apparatus according to example 8, wherein the coaptation body is a block.

Example 12. The apparatus according to example 8, wherein the coaptation body comprises a resilient frame.

Example 13. The apparatus according to example 12, wherein the patch comprises a sheet that covers frame.

Example 14. The apparatus according to example 8, wherein the implant comprises a sheet that defines a surface of the patch and a surface of the coaptation body.

Example 15. The apparatus according to example 8, wherein the coaptation body defines a hollow therein.

Example 16. The apparatus according to example 15, wherein the delivery tool is configured to inflate the coaptation body with a liquid.

Example 17. The apparatus according to example 15, wherein the coaptation body is configured to encourage fibrosis inside the hollow.

Example 18. The apparatus according to any one of examples 1-17, wherein the patch is self-expandable, such that the patch is adapted to be transluminally delivered, via the delivery tool, in a constrained state, and is biased to transition, from the constrained state, to a deployed state in which it is dimensioned to bridge the gap.

Example 19. The apparatus according to any one of examples 1-18, wherein:
the surface of the first leaflet is an atrial surface of the first leaflet,
the surface of the second leaflet is an atrial surface of the second leaflet, and
the delivery tool is adapted to secure the patch against the atrial surface of the first leaflet and the atrial surface of the second leaflet such that the patch bridges the gap.

Example 20. The apparatus according to any one of examples 1-19, wherein:
the surface of the first leaflet is a ventricular surface of the first leaflet,
the surface of the second leaflet is a ventricular surface of the second leaflet, and
the delivery tool is adapted to secure the patch against the ventricular surface of the first leaflet and the ventricular surface of the second leaflet such that the patch bridges the gap.

Example 21. The apparatus according to example 20, wherein:
the patch defines a plurality of slots along an edge of the patch, and
the delivery tool is adapted to secure the patch against the ventricular surface of the second leaflet such that a chorda of the valve is disposed within a slot of the plurality of slots.

Example 22. The apparatus according to any one of examples 1-21, wherein:
the first anchor is a first toggle anchor,
the second anchor is a second toggle anchor, and
the delivery tool is adapted to secure the patch against the surface of the first leaflet by driving the first toggle anchor through the first leaflet such that the first toggle anchor becomes disposed against an opposite surface of the first leaflet, and
the delivery tool is adapted to secure the patch against the surface of the second leaflet by driving the second toggle anchor through the second leaflet such that the second toggle anchor becomes disposed against an opposite surface of the second leaflet.

Example 23. The apparatus according to example 22, wherein:
the first toggle anchor is attached to the patch via a first cord, and
the second toggle anchor is attached to the patch via a second cord.

Example 24. The apparatus according to any one of examples 1-23, wherein:
the gap is a gap between the first leaflet, the second leaflet, and a third leaflet of the valve,
the implant further comprises a third anchor, and
the delivery tool is further adapted to secure the patch against a surface of the third leaflet using the third anchor, such that the patch bridges the gap between the first leaflet, the second leaflet, and the third leaflet.

Example 25. The apparatus according to example 24, wherein the delivery tool is adapted to secure the patch against the surface of the third leaflet such that the first leaflet, the second leaflet, and the third leaflet form more than one discrete orifice via which, during ventricular diastole, blood can flow into the ventricle.

Example 26. The apparatus according to example 25, wherein the delivery tool is adapted to secure the patch against the surface of the third leaflet such that the leaflets form three discrete orifices at the valve.

Example 27. The apparatus according to any one of examples 1-26, wherein the patch comprises a flexible sheet.

Example 28. The apparatus according to example 27, wherein the flexible sheet is impermeable.

Example 29. The apparatus according to example 27, wherein the flexible sheet is semi-impermeable.

Example 30. The apparatus according to example 27, wherein the flexible sheet comprises a fabric.

Example 31. The apparatus according to example 27, wherein the flexible sheet comprises a film.

Example 32. The apparatus according to example 27, wherein the patch comprises a frame, adapted to support the flexible sheet.

Example 33. The apparatus according to example 32, wherein the frame extends along a perimeter of the flexible sheet.

Example 34. The apparatus according to any one of examples 1-33, wherein the delivery tool is adapted to deliver the implant to the heart, and comprises:
a shaft, and
a first channel and a second channel, coupled to the shaft, each of the first and second channels adapted to house a respective one of the first and second anchors during delivery of the implant to the heart.

Example 35. The apparatus according to example 34, wherein the delivery tool has a delivery state in which the delivery tool is adapted to deliver the implant to the heart, and in which:
the channels are positioned medially, and
the patch is wrapped around a distal end of the shaft, such that the patch forms a cup-like shape around the channels and the shaft.

Example 36. The apparatus according to example 35, wherein:
the delivery tool has a deployment state in which the delivery tool is configured to anchor the first and second anchors to the first and second leaflets, and
the delivery tool is transitionable from the delivery state toward the deployment state by deflecting the first and second channels laterally with respect to the shaft.

Example 37. The apparatus according to example 35, wherein, for each of the first anchor and the second anchor, the respective channel has an axial slit that accommodates a cord extending from the anchor to the patch.

Example 38. The apparatus according to example 37, wherein:
the delivery tool is adapted to secure the patch against the surface of the first leaflet and the surface of the second leaflet by:
positioning the first channel against the first leaflet and second channel against the second leaflet, and
advancing each of the first and second anchors out of the respective channel and through the respective leaflet.

Example 39. The apparatus according to example 38, wherein the apparatus is configured such that, for each of the first and second anchors, advancing of the anchor out of the respective channel is facilitated by the respective cord sliding along the respective axial slit.

Example 40. An apparatus for repairing a valve of a heart, the heart having an atrium, a ventricle, the valve disposed therebetween, the apparatus comprising:
an implant, comprising:
a flexible patch;
a coaptation body, attached to the patch;
a first anchor; and
a second anchor; and
a delivery tool adapted to:
position the patch in the heart, and secure the patch against a surface of a first leaflet of the valve using the first anchor, and against a surface of a second leaflet of the valve using the second anchor, such that the coaptation body extends between the first leaflet and the second leaflet.

Example 41. The apparatus according to example 40, wherein the apparatus is sterilized.

Example 42. The apparatus according to any one of examples 40-41, wherein the coaptation body has a surface configured to encourage adhesion of the first leaflet and the second leaflet thereto.

Example 43. The apparatus according to any one of examples 40-42, wherein the implant comprises a resilient frame that defines at least part of the patch and at least part of the coaptation body.

Example 44. The apparatus according to any one of examples 40-43, wherein the coaptation body is a block.

Example 45. The apparatus according to any one of examples 40-44, wherein the implant comprises a sheet that defines a surface of the patch and a surface of the coaptation body.

Example 46. The apparatus according to any one of examples 40-45, wherein the coaptation body comprises a resilient frame.

Example 47. The apparatus according to example 46, wherein the patch comprises a sheet that covers frame.

Example 48. The apparatus according to any one of examples 40-47, wherein the coaptation body defines a hollow therein.

Example 49. The apparatus according to example 48, wherein the delivery tool is configured to inflate the coaptation body with a liquid.

Example 50. The apparatus according to example 48, wherein the coaptation body is configured to encourage fibrosis inside the hollow.

Example 51. A method of repairing a heart of a subject, the method comprising:
transluminally delivering an implant to the heart, the implant including a patch, a first anchor, and a second anchor;
using the first anchor, securing the patch against a surface of a first leaflet of a tricuspid valve of the heart; and
using the second anchor, securing the patch against a surface of a second leaflet of the tricuspid valve such that the patch bridges a gap between the first leaflet and the second leaflet.

Example 52. The method according to example 51, wherein securing the patch against the surface of the second leaflet comprises:
driving the second anchor through the second leaflet, such that a cord extends, from the second anchor, through the second leaflet, and towards the patch, and subsequently, advancing the patch over and along the cord toward the second anchor.

Example 53. The method according to any one of examples 51-52, wherein:
the implant further includes a coaptation body attached to the patch, and
securing the patch against the surface of the second leaflet comprises securing the patch such that the coaptation body extends into the gap.

Example 54. The method according to example 53, wherein:
the patch defines a patch plane, and
securing the patch such that the coaptation body extends into the gap comprises securing the patch such that the coaptation body extends orthogonally away from the patch plane and into the gap.

Example 55. The method according to example 53, wherein:
transluminally delivering the implant to the heart comprises transluminally delivering the implant to the heart while the coaptation body is in a compressed state, and
the method further comprises expanding the coaptation body within the heart.

Example 56. The method according to example 55, wherein expanding the coaptation body within the heart comprises inflating the coaptation body within the heart.

Example 57. The method according to example 55, wherein:
transluminally delivering the implant to the heart comprises transluminally delivering the implant to the heart while the coaptation body is constrained in the compressed state, and
expanding the coaptation body within the heart comprises releasing the coaptation body to automatically expand within the heart.

Example 58. The method according to example 53, wherein:
the coaptation body is a panel, and
securing the patch such that the coaptation body extends into the gap comprises securing the patch such that the panel extends into the gap.

Example 59. The method according to example 53, wherein:
the coaptation body is a spacer, and
securing the patch such that the coaptation body extends into the gap comprises securing the patch such that the spacer extends into the gap.

Example 60. The method according to example 53, wherein:
the coaptation body is a block, and
securing the patch such that the coaptation body extends into the gap comprises securing the patch such that the block extends into the gap.

Example 61. The method according to any one of examples 51-60, wherein:
securing the patch against the surface of the first leaflet comprises driving the first anchor through the first leaflet, and
securing the patch against the surface of the second leaflet comprises driving the second anchor through the second leaflet and into tissue of the second leaflet.

Example 62. The method according to any one of examples 51-61, wherein the method comprises covering the gap with the patch without eliminating the gap.

Example 63. The method according to any one of examples 51-62, wherein the method comprises covering the gap with the patch such that the tricuspid valve is bicuspidized.

Example 64. The method according to any one of examples 51-63, wherein:
the surface of the first leaflet is an atrial surface of the first leaflet,
the surface of the second leaflet is an atrial surface of the second leaflet, and
securing the patch against the surface of the second leaflet comprises securing the patch against the atrial surface of the second leaflet such that, within a right atrium of the heart, the patch bridges the gap between the first leaflet and the second leaflet.

Example 65. The method according to any one of examples 51-64, wherein:
the surface of the first leaflet is a ventricular surface of the first leaflet,
the surface of the second leaflet is a ventricular surface of the second leaflet, and
securing the patch against the surface of the second leaflet comprises securing the patch against the ventricular surface of the second leaflet.

Example 66. The method according to example 65, wherein:
the patch defines a plurality of slots along an edge of the patch, and
securing the patch against the ventricular surface of the second leaflet comprises securing the patch against the ventricular surface of the second leaflet such that a chorda of the tricuspid valve is disposed within a slot of the plurality of slots.

Example 67. The method according to any one of examples 51-66, wherein:
the first anchor is a first toggle anchor,
the second anchor is a second toggle anchor, and
securing the patch against the surface of the first leaflet comprises driving the first toggle anchor through the first leaflet such that the first toggle anchor becomes disposed against an opposite surface of the first leaflet, and
securing the patch against the surface of the second leaflet comprises driving the second toggle anchor through the second leaflet such that the second toggle anchor becomes disposed against an opposite surface of the second leaflet.

Example 68. The method according to example 67, wherein:
the first toggle anchor is attached to the patch via a first cord,
the second toggle anchor is attached to the patch via a second cord,
driving the first toggle anchor through the first leaflet comprises driving the first toggle anchor through the first leaflet such that the first cord extends, from the first anchor, through the first leaflet, to the patch, and
driving the second toggle anchor through the second leaflet comprises driving the second toggle anchor through the second leaflet such that the second cord extends, from the second anchor, through the second leaflet, to the patch.

Example 69. The method according to any one of examples 51-68, wherein securing the patch against the surface of the second leaflet comprises securing the patch against the surface of the second leaflet such that the patch covers a commissure that is common to the first leaflet and the second leaflet.

Example 70. The method according to example 69, wherein securing the patch against the surface of the second leaflet comprises securing the patch against the surface of the second leaflet such that the patch does not contact a third leaflet of the tricuspid valve.

Example 71. The method according to example 69, wherein securing the patch against the surface of the second leaflet comprises securing the patch against the surface of the second leaflet such that, during ventricular systole, a lip of the patch contacts a third leaflet of the tricuspid valve.

Example 72. The method according to example 71, wherein securing the patch against the surface of the second leaflet comprises securing the patch against the surface of the second leaflet such that the lip of the patch extends into a right ventricle of the heart.

Example 73. The method according to any one of examples 51-72, wherein:
the implant further includes a third anchor, and
the method further comprises, using the third anchor, securing the patch against a surface of a third leaflet of the tricuspid valve such that the patch bridges a gap between the first leaflet, the second leaflet, and the third leaflet.

Example 74. The method according to example 73, wherein securing the patch against the surface of the third leaflet comprises securing the patch against the surface of the third leaflet such that the first leaflet, the second leaflet, and the third leaflet form more than one discrete orifice via which, during ventricular diastole, blood can flow into a right ventricle of the heart.

Example 75. The method according to example 74, wherein securing the patch against the surface of the third leaflet comprises securing the patch against the surface of the third leaflet such that the leaflets form three discrete orifices at the valve.

Example 76. The method according to example 51, further comprising:
sterilizing the implant prior to delivering the implant to the heart.

Example 77. A method for repairing a heart of a subject, the heart having an atrium, a ventricle, and a valve therebetween, the method comprising, from the ventricle:
transluminally attaching a first anchor to a first leaflet of the valve while the first anchor is attached to a first cord,
transluminally attaching a second anchor to a second leaflet of the valve while the second anchor is attached to a second cord,
while a patch is threaded onto the first cord and the second cord, transluminally advancing the patch over and along the first and second cords towards the valve, and
subsequently, securing the patch to a surface of the first leaflet and a surface of the second leaflet by fastening the patch to the first and second cords.

Example 78. The method according to example 77, wherein the valve is a bicuspid valve, and wherein securing the patch to the surface of the first leaflet and the surface of the second leaflet comprises securing the patch to the surface of the first leaflet and the surface of the second leaflet of the bicuspid valve.

Example 79. The method according to any one of examples 77-78, wherein the valve is a tricuspid valve, and wherein securing the patch to the surface of the first leaflet and the surface of the second leaflet comprises securing the patch to the surface of the first leaflet and the surface of the second leaflet of the tricuspid valve.

Example 80. The method according to any one of examples 77-79, wherein the method further comprises:
transluminally attaching a third anchor to a third leaflet of the valve while the third anchor is attached to a third cord,
while the patch is threaded onto the third cord, transluminally advancing the patch over and along the third cord towards the valve, and securing the patch to the third leaflet by fastening the patch to the third cord.

Example 81. The method according to any one of examples 77-80, wherein:
the ventricle is a right ventricle,
the surface of the first leaflet is a ventricular surface of the first leaflet,
the surface of the second leaflet is a ventricular surface of the second leaflet, and
securing the patch to the surface of the second leaflet comprises securing the patch to the surface of the second leaflet such that, within the right ventricle, the patch bridges a gap between the first leaflet and the second leaflet.

Example 82. The method according to example 81, wherein:
the patch defines a plurality of slots along an edge of the patch, and
securing the patch to the surface of the second leaflet comprises securing the patch to the surface of the second leaflet such that, within the right ventricle, a chorda of the valve is disposed within a slot of the plurality of slots.

Example 83. The method according to example 77, further comprising sterilizing the first anchor, the second anchor, the first cord, the second cord and the patch prior to delivering the first anchor, the second anchor, the first cord, the second cord and the patch to the heart of the subject.

Example 84. A method of repairing a valve of a heart of a subject, the method comprising:
transluminally delivering an implant to the heart, the implant including a patch, a coaptation body attached to the patch, and an anchor; and
securing the patch against a surface of a first leaflet of the valve and against a surface of a second leaflet of the valve using at least the anchor, such that the coaptation body extends between the first leaflet and the second leaflet.

Example 85. The method according to example 84, wherein:
the valve is disposed between an atrium and a ventricle of the heart, and
securing the patch against the surface of the first leaflet and against the surface of the second leaflet such that the coaptation body extends between the first leaflet and the second leaflet, comprises securing the patch against an atrial surface of the first leaflet and against an atrial surface of the second leaflet such that the coaptation body extends between the first leaflet and the second leaflet toward the ventricle.

Example 86. The method according any one of examples 84-85, wherein the valve is a tricuspid valve, and wherein securing the patch to the surface of the first leaflet of the valve comprises securing the patch to a surface of a first leaflet of the tricuspid valve.

Example 87. The method according to any one of examples 84-86, wherein:
the patch defines a patch plane, and
securing the patch to the surface of the first leaflet and the surface of the second leaflet comprises securing the patch such that the coaptation body extends orthogonally away from the patch plane and towards a ventricle downstream of the valve.

Example 88. The method according to any one of examples 84-87, wherein:
the coaptation body is a panel, and
securing the patch to the surface of the first leaflet and the surface of the second leaflet comprises securing the patch such that the panel extends into a gap between the first leaflet and the second leaflet.

Example 89. The method according to any one of examples 84-87, wherein:
the coaptation body is a spacer, and
securing the patch to the surface of the first leaflet and the surface of the second leaflet comprises securing the patch such that the spacer extends into a gap between the first leaflet and the second leaflet.

Example 90. The method according to any one of examples 84-87, wherein:
the coaptation body is a block, and
securing the patch such to the surface of the first leaflet and the surface of the second leaflet comprises securing the patch such that the block extends into a gap between the first leaflet and the second leaflet.

Example 91. The method according to any one of examples 84-90, wherein:
transluminally delivering the implant to the heart comprises transluminally delivering the implant to the heart while the coaptation body is in a compressed state, and
the method further comprises expanding the coaptation body within the heart.

Example 92. The method according to example 91, wherein expanding the coaptation body within the heart comprises inflating the coaptation body within the heart.

Example 93. The method according to example 91, wherein:
transluminally delivering the implant to the heart comprises transluminally delivering the implant to the heart while the coaptation body is constrained in the compressed state, and
expanding the coaptation body within the heart comprises releasing the coaptation body to automatically expand within the heart.

Example 94. The method according to any one of examples 84-93, further comprising sterilizing the implant prior to delivering the implant to the heart.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the treatment techniques, methods, steps, etc. described or suggested herein or references incorporated herein can be performed on a living animal or on a non-living simulation, such as on a cadaver, cadaver heart, simulator (e.g., with the body parts, tissue, etc. being simulated), etc.

What is claimed is:

1. Apparatus for repairing a valve situated between an atrium and a ventricle of a heart of a subject, the valve having a first leaflet and a second leaflet, the apparatus comprising:
an implant, comprising:
a flexible patch defining slots along an edge of the patch;
a first anchor; and
a second anchor; and
a delivery tool adapted to:
transluminally deliver the implant to the heart; and
implant the implant in the ventricle by securing the patch against a ventricular surface of the first leaflet by anchoring the first anchor to the first leaflet, and against a ventricular surface of the second leaflet by anchoring the second anchor to the second leaflet, such that:
chordae of the valve extend through the slots, and
the patch bridges a gap between the first leaflet and the second leaflet.

2. The apparatus according to claim 1, wherein:
the valve is a tricuspid valve, and
the delivery tool is adapted to implant the implant in the ventricle such that the tricuspid valve is bicuspidized.

3. The apparatus according to claim 1, wherein the delivery tool is adapted to implant the implant in the ventricle such that the patch covers a commissure that is common to the first leaflet and the second leaflet.

4. The apparatus according to claim 1, wherein:
the delivery tool further comprises a reference support adapted to apply a reference force against an atrial surface of the first leaflet, and
the delivery tool is adapted to secure the patch against the ventricular surface of the first leaflet by driving the first anchor through the first leaflet while the reference support is pressed against the atrial surface of the first leaflet.

5. The apparatus according to claim 1, wherein the implant further comprises a coaptation body, attached to the patch, and wherein the delivery tool is adapted to secure the patch such that the coaptation body extends into the gap.

6. The apparatus according to claim 5, wherein the coaptation body has a surface configured to encourage adhesion of the first leaflet and the second leaflet thereto.

7. The apparatus according to claim 5, wherein the coaptation body comprises a resilient frame.

8. The apparatus according to claim 7, wherein the patch comprises a sheet that covers frame.

9. The apparatus according to claim 5, wherein the coaptation body defines a hollow therein.

10. The apparatus according to claim 9, wherein the delivery tool is configured to inflate the coaptation body with a liquid.

11. The apparatus according to claim 9, wherein the coaptation body is configured to encourage fibrosis inside the hollow.

12. The apparatus according to claim 1, wherein:
the first anchor is a first toggle anchor,
the second anchor is a second toggle anchor, and
the delivery tool is adapted to anchor the first anchor to the first leaflet by driving the first toggle anchor through the first leaflet such that the first toggle anchor becomes disposed against an atrial surface of the first leaflet, and
the delivery tool is adapted to anchor the second anchor to the second leaflet by driving the second toggle anchor through the second leaflet such that the second toggle anchor becomes disposed against an atrial surface of the second leaflet.

13. The apparatus according to claim 1, wherein:
the gap is between the first leaflet, the second leaflet, and a third leaflet of the valve,
the implant further comprises a third anchor, and
the delivery tool is adapted to implant the patch in the ventricle further by securing the patch against a ventricular surface of the third leaflet by anchoring the third anchor to the third leaflet, such that the patch bridges the gap between the first leaflet, the second leaflet, and the third leaflet.

14. The apparatus according to claim 13, wherein the delivery tool is adapted to implant the patch in the ventricle such that the first leaflet, the second leaflet, and the third leaflet form three discrete orifices via which, during ventricular diastole, blood can flow into the ventricle.

15. The apparatus according to claim 1, wherein the patch comprises a flexible sheet.

16. The apparatus according to claim 15, wherein the patch comprises a frame that extends along a perimeter of the flexible sheet and that is adapted to support the flexible sheet.

17. The apparatus according to claim 1, wherein the delivery tool comprises:
a shaft, and
a first channel and a second channel, coupled to the shaft, each of the first and second channels adapted to house a respective one of the first and second anchors during transluminal delivery of the implant to the heart.

18. The apparatus according to claim 17, wherein the delivery tool has a delivery state in which the delivery tool is adapted to deliver the implant to the heart, and in which:
the first and second channels are positioned medially, and
the patch is wrapped around a distal end of the shaft, such that the patch forms a cup-like shape around the channels and the shaft.

19. The apparatus according to claim 18, wherein:
the delivery tool has a deployment state in which the delivery tool is configured to anchor the first and second anchors to the first and second leaflets, and
the delivery tool is transitionable from the delivery state toward the deployment state by deflecting the first and second channels laterally with respect to the shaft.

20. A method for repairing a valve situated between an atrium and a ventricle of a heart of a subject, the valve having a first leaflet and a second leaflet, the method comprising:
using a delivery tool, transluminally delivering an implant to the heart, the implant including:
a flexible patch defining slots along an edge of the patch;
a first anchor; and
a second anchor; and
using the delivery tool, implanting the implant in the ventricle by securing the patch against a ventricular surface of the first leaflet by anchoring the first anchor to the first leaflet, and against a ventricular surface of the second leaflet by anchoring the second anchor to the second leaflet, such that:
chordae of the valve extend through the slots, and
the patch bridges a gap between the first leaflet and the second leaflet.
